(19) United States
(12) Patent Application Publication
(10) Pub. No.: US 2025/0009268 A1
(43) Pub. Date: Jan. 9, 2025

(54) PHYSIOLOGICAL DIAGNOSIS INTERFACE

(71) Applicant: KING FAISAL UNIVERSITY, Al-Ahsa (SA)

(72) Inventors: Surbhi BHATIA, Al-Ahsa (SA); Pragyaditya DAS, Al-Ahsa (JP)

(21) Appl. No.: 18/218,648

(22) Filed: Jul. 6, 2023

(51) Int. Cl. A61B 5/16; A61B 5/00; A61B 5/0205; A61B 5/145

(52) U.S. Cl. CPC A61B 5/165; A61B 5/0006; A61B 5/0205; A61B 5/14517; A61B 5/14532; A61B 5/6825; A61B 5/7264; A61B 5/7455

(57) ABSTRACT

A physiological diagnosis system includes a semi permeable membrane having one or more sensors; an accumulator that receives data from the one or more sensors; and a machine learning engine that analyzes the data from the accumulator and provides a diagnosis based on analysis of the data.

### BACKGROUND

### 1. Field

The present disclosure relates to non-invasive physiological data systems for diagnosing a patient, and particularly to non-invasive physiological data systems that diagnose a patient for conditions such as anxiety, depression, etc.

### 2. Description of the Related Art

Mental health has been a subject of great interest in recent years. The COVID-19 pandemic created more clarity amongst the masses regarding the topic of mental health. There are reported burn-outs and frustration in people from all sectors and age-groups. Though help is available, the number of people with mental health concerns is still high due to either medical non-adherence or to people not acknowledging that they need help.

### SUMMARY

Human beings respond well to what can be seen and what can be touched. A non-invasive method which employs any or all of Blood Pressure, SpO2, Blood Sugar Levels, Hemoglobin, etc. to create a Data Science model can measure a person's health parameters in a relatively easy way.

Based on the output, a mechanical hand made of medical grade polymers will go towards a person for a handshake—the tonality, skin temperature, warmness, etc. of the mechanical hand will be based on how the person is feeling at the time. This will give a physical human-based feedback to the person.

A physiological diagnosis system, in one embodiment, includes: a semi permeable membrane having one or more sensors; an accumulator that receives data from the one or more sensors; and a machine learning engine that analyzes the data from the accumulator and provides a diagnosis based on analysis of the data.

The one or more sensors includes at least a cortisol sensor made of a nano-porous flexible electrode system. The nano-porous flexible electrode system is made of molybdenum di-sulfide.

The one of more sensors includes an SpO2 and heartbeat sensor.

The one or more sensors includes a blood sugar sensor.

The one or more sensors includes an EEG and blood pressure sensor.

The system can further include: a first feedback loop that provides feedback in the form of an anxiety score to the machine learning engine; and a second feedback loop that provides physical feedback to the patient through the modification of temperature and tonality of the semi permeable membrane.

A physiological diagnosis method includes: receiving biomedical data from a user through a semi permeable membrane having one or more sensors; accumulating the biomedical data in an accumulator that receives the biomedical data from the one or more sensors; analyzing the biomedical data in a machine learning engine that analyzes the biomedical data accumulated in the accumulator; and providing a diagnosis based on the analysis from the machine learning engine.

The one or more sensors, from which the biomedical data is received, includes a cortisol sensor made of a nano-porous flexible electrode system. The nano-porous flexible electrode system is made of molybdenum di-sulfide.

The one of more sensors, from which the biomedical data is received, includes an SpO2 and heartbeat sensor.

The one or more sensors, from which the biomedical data is received, includes a blood sugar sensor.

The one or more sensors, from which the biomedical data is received, includes an EEG and blood pressure sensor.

The method further includes: providing feedback, through a first feedback loop, in the form of an anxiety score to the machine learning engine; and providing physical feedback, through a second feedback loop, to the patient through the modification of temperature and tonality of the semi permeable membrane.

These and other features of the present subject matter will become readily apparent upon further review of the following specification.

### BRIEF DESCRIPTION OF THE DRAWINGS

FIG. 1 is an illustration of a physiological diagnosis interface.

FIG. 2 is an illustration of the machine learning engine.

FIG. 3 is a flow diagram of a physiological diagnosis method

Similar reference characters denote corresponding features consistently throughout the attached drawings.

### DETAILED DESCRIPTION OF THE PREFERRED EMBODIMENTS

For the first time, a person's mental condition can be translated using Data Science concepts using humanoid feedback. Use of Human-to-Humanoid Behavioral Integration is now possible.

An electronic unit can be used to measure a person's health parameters in a relatively short period of time. A person can place her hand on a semi-permeable membrane and health parameters are measured using reflectance.

This system is caste/gender/race/age agnostic. It can be used by all and does not require any disposable items other than a semi-permeable membrane.

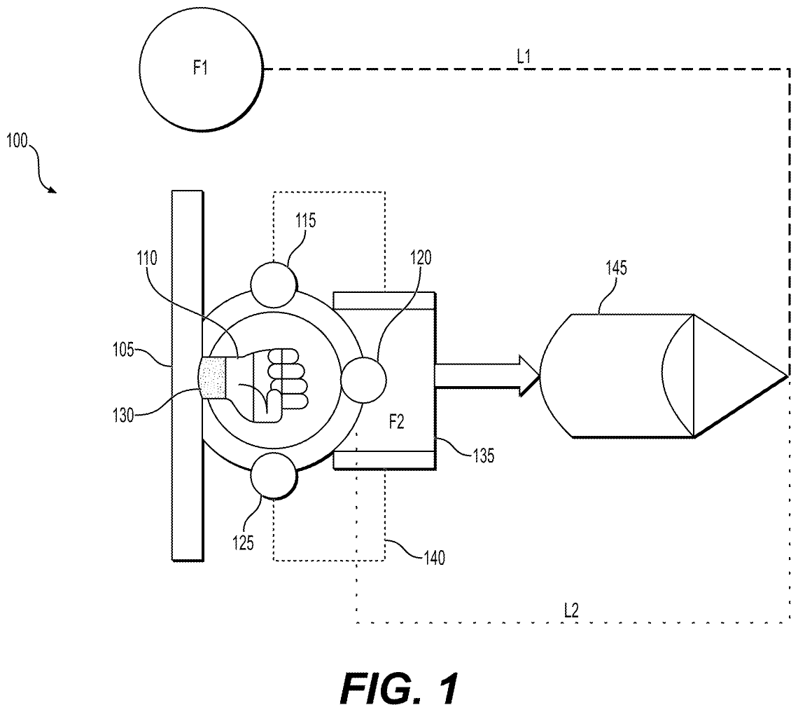

FIG. 1 is an illustration of a physiological diagnosis interface 100. It includes an interface 105, which is a human/machine interface that is used to collect data on the user. The interface 105 includes a semi permeable membrane 110, which a user physically touches. The semi permeable membrane 110 is a biologically stable polymer and can change its parameters to match the physical conditions of a person. This provides more intuitive feedback to the user.

In FIG. 1, the semi permeable membrane 110 is in the shape of a human hand. The membrane exposes a user's skin to a sensing unit which employs various sensors 115,120,125,130 to capture several biomedical indicators to detect anxiety.

Cortisol sensor 115 measures cortisol levels in blood. Cortisol is a stress hormone in the human body. This sensor is made of a nano-porous flexible electrode system made of molybdenum di-sulfide. Cortisol sensor 115 measures the Cortisol from traces of sweat from the user's hand (8.16 to 141.7 ng/ml-mild to very high stress indicators). In cases where there is no sweat present, gradual and gentle heating is made to the semi permeable membrane to generate sweat in the hand of the user, thereby allowing cortisol sensor 115 to measure the cortisol levels.

SpO2 and heartbeat sensor 120 measures the SpO2 and heartbeat of a user. It is known that the right-hand thumb is statistically the best finger for measuring SpO2 and a heartbeat. It is advantageous if the SpO2 and heartbeat sensor 120 comes in contact with the right-hand thumb of the user to take these measurements.

Blood sugar sensor 125 measures carbohydrate levels in the blood of the user. Anxiety often leads to “Anxiety induced” carbohydrate overdose. This sensor will indicate this parameter by measuring the blood sugar levels and determining if the levels are high for the user.

EEG and blood pressure sensor 130 measures brain and blood pressure parameters. Studies have shown that anxiety does not have any effect on long term high blood pressure in humans, however short episodes of harmful high blood pressures are seen in people with anxiety. EEG signals can be used to detect anxiety. In the EEG and blood pressure sensor 130, anxiety is measured as a factor which is inversely related to the asymmetry of the frontal alpha band.

An accumulator 135 gathers data taken from the sensors 115,120,125,130. The data is then passed through a convolution layer 140. A machine learning engine 145 conducts feature extraction on the data gathered by accumulator 135, which is then passed through the convolution layer 140. The data is further analyzed and a result is output to the user in the form of a change in the temperature and/or tonality of the semi permeable membrane.

Feedback loop 1 (L1) provides feedback in the form of an anxiety score (F1) on the basis of various biomedical indicators and is presented on the Hamilton Anxiety Scale.

Feedback loop 2 (L2) provides physical feedback (F2) to the user through the modification of temperature and tonality of the semi permeable membrane 110. This is where the system starts depicting a human type of system, and therefore justifies the nomenclature “humanoid”.

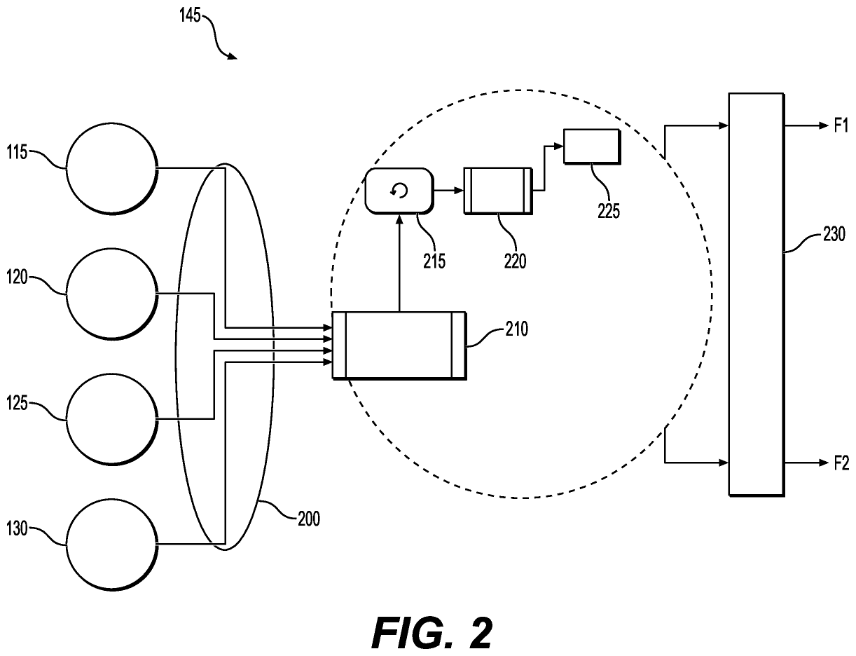

FIG. 2 is an illustration of the machine learning engine 145. In this design, data is collected from sensors 115, 120, 125 and 130. Data analysis is conducted by passing data through a convolutional layer 200 which squashes the vector matrixes for easier analysis. The data is then passed through an ensemble learning system 210, where the data is run n times with corrected weights, getting inside the next ensemble. Testing 215 is repeated giving an error 220 and weighting the error 225 to further minimize error.

The testing data units are matched, and then an output is transmitted to the output distribution box 230 through which there are two feedbacks given as F1 and F2, (which are feedback systems), as seen in the distribution table. F1 can be in the form of an anxiety score, and F2 can be a physical feedback to the user through the modification of temperature and tonality of the semi permeable membrane 110.

As can be seen from the datasets that are used in the system, the features and data points are highly associative in nature. To see this from a computational point of view, high associativity of the information are interpreted as nodes in a high dimensional graph, in which the structure of the graph changes with changes in each node. Therefore, a Convolutional Graph Network is used, which is a semi supervised classification technique where a loss-less scaling of the system is needed. Loss-less scaling is meant that the system can be scaled to very large use cases with minimal losses in terms of computational and system accuracy.

The vector matrices are column matrices which are often termed as “Vectors” in the machine learning field. These vector matrices are feature vectors that are used as a feed to the Convolutional Graph Network for classification purposes.

Data collection by sensors, in one example, is stored in matrices of n-dimensions. For example:

$$M = \begin{matrix}[ {a}_{11} … {a}_{nm} \\ {b}_{11} … \\ ⋮ \\ {z}_{11} … {z}_{nm} ] n \times n\end{matrix}$$

The above matrix M is then passed through various steps to create a “Convolution Graph,” which is a relational matrix called M.

Matrix manipulations are again performed on M, to create vector matrices, which help in accessing features of the input data. M is then broken into individual vector matrices→Mᵥ₁′, Mᵥ₂′, etc. as shown below.

$${M}_{v 1}^{′} = [ \begin{matrix}a 11 \\ b 11 \\ ⋮\end{matrix} ]$$

Ensemble learning is then performed using various ML methods. The ML methods are put together and their joint best is considered as the output.

$$R 1 = \begin{matrix}f ( {M}_{v 1}^{′} , {M}_{v 2}^{′} , {M}_{vn}^{′} ) \\ Linear Regression\end{matrix} . \\ R 2 = \begin{matrix}f ( {M}_{v 1}^{′} , {M}_{v 2}^{′} , {M}_{vn}^{′} ) \\ Logistic Regression\end{matrix} . \\ R 3 = \begin{matrix}f ( {M}_{v 1}^{′} , {M}_{v 2}^{′} , {M}_{vn}^{′} ) \\ SVM\end{matrix} .$$

The outputs are then averaged for accuracy through a bagging procedure, also known as bootstrap aggregation.

Accuracy Model=Arg(R1,R2,R3)

The problem solved by the present system is unique. There is practically no previous work done on Anxiety detection from biological markers using data science techniques. It has been, therefore, impossible to ascertain which models will work best for the system. In situations where the best fit model for machine learning is not readily known, the ensemble learning methodology is used. In this methodology, multiple machine learning models are used together, and the “combined-best” accuracy is achieved from the system. In this way, the present system derives the best out of multiple models.

It is interesting to note that Ensemble learning is used inside a CGN architecture. Multiple CGN instances are created, which are fed inside an Ensemble learning system, which is then sent to a data testing loop. Based on the error of the output, a feedback loop gives information to the ensemble learning to modify its weights.

Testing of data units is done by comparing the gold standard (“known correct” value) with the output generated by the system.

This feedback system is implemented after the ensemble layer of the system. This is done to correct the ensemble layer weights on a real time basis to achieve maximum accuracy.

In short, data is sent from different sub-systems to the CGN layer, which the data is presented in the form of an associative graph. This graph then transmits the data to the Ensemble learning model. The output of this model is checked against a “test” data set, based on the error reported. A feedback loop sends a trigger to the Ensemble learning model to make changes in its model weights. This iteration is done for n-number of times till we reach an accuracy of >99%.

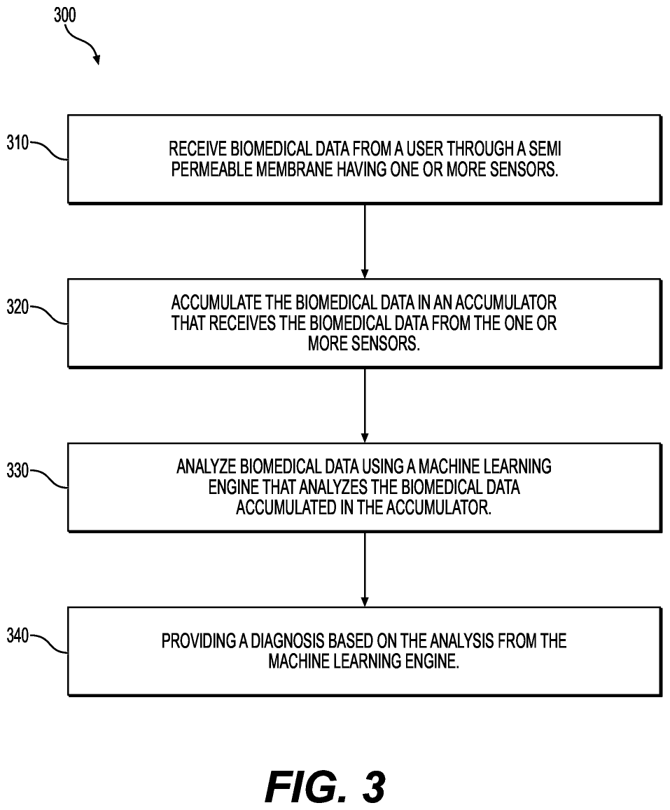

FIG. 3 is a flow diagram of a physiological diagnosis method 300. The method begins with box 310 where biomedical data is received from a user through a semi permeable membrane having one or more sensors. In box 320 the biomedical data is accumulated in an accumulator that receives the biomedical data from the one or more sensors. The biomedical data is analyzed in box 330 using a machine learning engine that analyzes the biomedical data accumulated in the accumulator. A diagnosis based on the analysis from the machine learning engine is provided in box 340.

A first feedback loop can provide feedback in the form of an anxiety score to the machine learning engine.

A second feedback loop can provide physical feedback to the patient through the modification of temperature and tonality of the semi permeable membrane.

The one or more sensors, from which the biomedical data is received, includes a cortisol sensor made of a nano-porous flexible electrode system.

The nano-porous flexible electrode system is made of molybdenum di-sulfide.

The one of more sensors, from which the biomedical data is received, includes an SpO2 and heartbeat sensor.

The one or more sensors, from which the biomedical data is received, includes a blood sugar sensor.

The one or more sensors, from which the biomedical data is received, includes an EEG and blood pressure sensor.

It is to be understood that the system and method for providing a physiological diagnosis is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A physiological diagnosis system comprising:
a semi permeable membrane having one or more sensors;
an accumulator that receives data from the one or more sensors; and
a machine learning engine that analyzes the data from the accumulator and provides a diagnosis based on analysis of the data,
wherein the semi permeable membrane is in a form of a graspable humanoid hand which is configured to be graspable by a right hand of a patient, and wherein the one or more sensors are configured to take a sensor measurement from a right hand thumb of the patient.

2. The system as recited in claim 1, wherein the one or more sensors comprises a cortisol sensor made of a nano-porous flexible electrode system.

3. The system as recited in claim 2, wherein the nano-porous flexible electrode system is made of molybdenum di-sulfide.

4. The system as recited in claim 1, wherein the one or more sensors comprises an SpO₂ and heartbeat sensor.

5. The system as recited in claim 1, wherein the one or more sensors comprises a blood sugar sensor.

6. The system as recited in claim 1, wherein the one or more sensors comprises an EEG and blood pressure sensor.

7. (canceled)

8. A physiological diagnosis method, comprising:
receiving biomedical data from a user through a semi permeable membrane having one or more sensors;
accumulating the biomedical data in an accumulator that receives the biomedical data from the one or more sensors;
analyzing the biomedical data in a machine learning engine that analyzes the biomedical data accumulated in the accumulator; and
providing a diagnosis based on the analysis from the machine learning engine,
wherein the semi permeable membrane is in a form of a graspable humanoid hand which is configured to be graspable by a right hand of a patient, and wherein the one or more sensors are configured to take a sensor measurement from a right hand thumb of the patient.

9. The method as recited in claim 8, wherein the one or more sensors, from which the biomedical data is received, comprises a cortisol sensor made of a nano-porous flexible electrode system.

10. The method as recited in claim 9, wherein the nano-porous flexible electrode system is made of molybdenum di-sulfide.

11. The method as recited in claim 1, wherein the one or more sensors, from which the biomedical data is received, comprises an SpO₂ and heartbeat sensor.

12. The method as recited in claim 8, wherein the one or more sensors, from which the biomedical data is received, comprises a blood sugar sensor.

13. The method as recited in claim 8, wherein the one or more sensors, from which the biomedical data is received, comprises an EEG and blood pressure sensor.

14. (canceled)
